(19) United States
(12) Patent Application Publication
(10) Pub. No.: US 2022/0257610 A1
(43) Pub. Date: Aug. 18, 2022

(54) Enhancing drug activity through accentuated buccal/sublingual administration.

(71) Applicant: Debasish Banerjee, Kolkata (IN)

(72) Inventor: Debasish Banerjee, Kolkata (IN)

(21) Appl. No.: 17/628,253

(22) PCT Filed: Jul. 28, 2019
(86) PCT No.: PCT/IB2019/056429

§ 371 Date: Jan. 19, 2022

(51) Int. Cl. A61K 31/5517; A61K 9/00; A61K 31/64; A61P 3/10; A61K 9/20

(52) U.S. Cl. CPC A61K 31/5517; A61K 9/006; A61K 9/2054; A61P 3/10; A61K 9/0056; A61K 31/64

(57) ABSTRACT

The invention describes an approach to sublingual administration of drugs where in pharmacologically inactive adjuvant have been used to transport the drugs rapidly to the tissue receptor sites to create a faster and a much greater response compared to oral administration or sublingual administration of the same drugs without the use of adjuvant. These compounds enhance the passage of the drug through the buccal mucosa and transfer to the active site. Here we have enhanced the activity of glipizide and alprazzolam by their use. In the first case the activity of glipizide is enhanced 32 times and in the 2_{nd} case the activity of alprazolam by 2 times approximately. An advantage of enhancing the activity glipizide is that it obliterates the use of metformin thereby making the product much safer for use and effective in situations of diabetic coma and lactic acidosis where mortality rates are around 60%.

### CROSS REFERENCE TO RELATED APPLICATIONS

International Application Number: PCT/IB 2019/056429—Int465-832, dated 28 Jul. 2019.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable.

### SEQUENCE LISTING

Not Applicable.

### STATEMENT REGARDING PRIOR DISCLOSURE BY INVENTOR OR JOINT INVENTOR

832/KOL/2009 dated May 9, 2009, Applicant/Inventor Debasish Banerjee, Patent granted. 465/KOL/2010 dated 26 Apr. 2010, Applicant/Inventor Debasish Banerjee, application pending, 61/KOL/2012 dated 24 Jan. 2012, Applicant/Inventor Debasish Banerjee, application pending. BACK

### GROUND OF THE INVENTION

Patent Classification A/61K 31/00

Increased activity at reduced dosages has been the endeavour of most drug researchers in the World. Increased therapeutic activity (compared to normal activity at presently used therapeutic doses) reduces the quantity of drug to be administered, thereby reducing the occurrence of side effects both on short term and long term use, and reduction of toxicity of drugs having low therapeutic indices. New vistas of treatment become available, where heretofore, the drug was not usable due to the high dosage required, which would render it toxic. Cost of treatment related to benefits, are also enormous, as reduction of doses lead to treatment cost reduction.

Heretofore drug absorption was considered to be 1^{st} order, which meant that the amount of drug absorbed into the system, and the resulting tissue concentration and pharmacological effect, was considered to be proportional to the amount of drug being administered. It was assumed that the greater the amount of drug being administered greater would be the amount transported to the receptor site and vice versa, which by and large was true. Later it became apparent that the pharmacological effect is more a function of the fraction of receptor sites occupied over a particular threshold and it is here this intervention works. Earlier amounts of drug being transported to the receptor depended on the physicochemical characteristics of the drug, solubility and dissolution rate diffusivity through biological membranes etc. Here we have invented a method which increases the transfer of drugs to the receptor site thereby increasing the receptor site (target organ) concentration, resulting in a rapid increase in receptor occupation even at lower doses, (as distinguished from membrane permeation enhancers), resulting in a faster onset and increased effectiveness of the drug.

### BRIEF SUMMARY OF THE INVENTION

Drug action is a function of the fraction of receptors occupied over a particular threshold. The actual fraction of drugs reaching the receptor site from a given administered dose depends on various ADME factors which slow down the transfer rate of drugs into the receptor site lowering receptor occupation at any given point in time. (To be noted here is that even large quantities of drug transferred slowly over extended time periods, so that the threshold receptor occupation rate is not crossed, would elicit no response.) This invention addresses the problem by the use of agents (adjuvant) which increase the transfer of drugs to the receptor site, increasing the receptor site drug concentration rapidly, lowering onset times and increasing effectiveness and duration of action so much so that drug doses need to be reduced or composition altered, in order to obtain an effect equivalent to an oral dose. The method has been successfully applied to two compounds in this patent application.

### BRIEF DESCRIPTION OF DRAWINGS

The application file contains at least one drawing executed in colour. Copies of this application publication with color drawings will be provided by the office on request and payment of the necessary fee.

FIG. 1 to 10A Refer to example 1 and FIG. 11 to 13 refer to example 2.

FIG. 1, X axis, Time in minutes, Y axis, Response in % of fasting blood sugar, mg/dL, Series-1, glucose base line; Ser-2, glipizide-2.5 mg; Ser-3, sublingual glipizide −2.5 mg with adjuvant.

FIG. 2, X axis, Time in minutes, Y axis, Response in % of fasting blood sugar, mg/dL, Series-3, glucose base line; Ser-1, glipizide-5.0 mg, Ser-2 sublingual glipizide −5.0 mg with adjuvant.

FIG. 3, X axis, Time in minutes, Y axis, Response in % of fasting blood sugar, mg/dL, Series-1 glucose base line, Ser-2 Amaryl MP-2, Ser-3 sublingual glipizide 5.0 mg with adjuvant.

FIG. 4, X axis, Time in minutes, Y axis, Response in % of fasting blood sugar, mg/dL, Series-1 glucose base line, Ser-2 glynase MF, Ser-3 sublingual glipizide 5.0 mg with adjuvant.

FIG. 5, X axis, log dose×100, Y axis, Response as measured by the reduction of blood sugar from the glucose base line of Series-1 glipizide oral, Ser-2 sublingual glipizide with adjuvant. (These differences are obtained from FIGS. 1 &2)

FIGS. 6 A&B, Duration of action of 7.5 mg of sublingual glipizide and adjuvant. X axis,time in minutes, Y axis response in mg/dL of blood sugar as a % age of fasting blood glucose. Fig A, immediately after the drug, fig B, after 12 hours. Series-1 glucose base line from FIG. 1, Ser-2, drug.

FIG. 7, Actual case study in a diabetic patient, X axis, time in minutes, Y axis blood sugar mg/dL

FIG. 8, Glucose tolerance test using 150 gm of glucose, and 5 mg of sublingual glipizide with adjuvant, X axis, time in minutes, Y axis, blood sugar in mg/dL

FIG. 9, Comparison of sublingual glipizide 7.5 mg with adjuvants, and gluconorm. X axis time in minutes, Y axis blood sugar in mg/dL % age of fasting blood sugar. Series-1 glucose base line, Ser-2, glipizide with adjuvant, Ser-3, gluconorm XL

FIG. 10, Comparison of sublingual glipizide 5.0 mg with adjuvant and sublingual glipizide 5.0 mg, Xaxis,time in minutes, Y axis, blood sugar in mg/dL as a % age of fasting blood sugar Series-1, glucose base line, Ser-2, sublingual glipizide 5.0 mg, Ser-3, sublingual glipizide 5.0 mg with adjuvants.

FIG. 10A, Effect of increase in quantity of adjuvant, X-axis time in minutes, Y-axis blood sugar in mg/dL as a % age of FBS, Series-1, glucose base line, Ser-2, sublingual glipizide 7.5 mg with 1× of adjuvant, Ser-3 sublingual glipizide 7.5 mg with 2× of adjuvant.

FIG. 11 Log dose (mcg/kg, x-axis) and response (sleep time, mins, y-axis) curve of orally administered Alprazolam in rabbits

FIG. 12 Log dose (mcg/kg, x-axis) and response (sleep time, mins, y-axis) comparison for Alprazolam in rabbits, Series-1 oral, Series-2 sublingual with adjuvant.

FIG. 13 Log dose (mcg/kg, x-axis) and response (sleep time, mins, y-axis) comparison for Alprazolam in humans, Series-1 oral, series-2 sublingual with adjuvant.

### DETAILED DESCRIPTION OF THE INVENTION

The method relates to enhancing the amount of drug that reaches the active site, that is receptor site concentration by increasing the rate of its transfer to the target organ. Historically some drugs, sorbitrate for instance have had lower sublingual dosage than oral dose. This is principally due to the fact that therapeutic action is a function of the amount of receptors (over a particular threshold) which are activated at that point of time. Earlier drug absorption was considered first order and hence increase in drug concentration (that is dose) led to increased absorption and receptor site transfer and thereby increase in the effect. This work provides evidence that transfer may be increased by the use of adjuvant without increasing the dose and that leads to increased receptor occupancy and increased effect, or, in other words, effect depends on receptor occupancy rather than drug concentration. This novel approach allows us to increase activity without dose increase and without subsequent increase in toxicity and untoward effects, and allows its use in novel methods of treatment for diseases considered untreatable earlier. For instance it forms the basis of treatment of diabetic ketoacidosis, for which there was no treatment except insulin, and mortality rates were as high as 60%. To be noted is that the same amount of drug if absorbed slowly over prolonged periods in time so that a subtherapeutic number of receptors are activated, it would elicit a much lower response if any at all. Sorbitrate due to its physicochemical nature is absorbed much faster sublingually than orally. Most drugs do not have such properties.

We have developed a process for enhancing the target organ permeability through buccal/sublingual absorption of drugs (increased receptor site concentration), by the use of added adjuvant so that the transfer of drugs are much greater and much faster, and thereby the drugs evoke a higher response and a faster onset of action over a longer duration of time, than conventional sub-lingual and oral dosage forms. To be noted here is that glipizide when orally administered is absorbed to the extent of 95% from the GI tract. Therefore had it been 100% absorbed the effect increase would have been only 5%. This 3200% increase is due to increased receptor occupation of the same or similar dose of the drug due to the effect of permeability enhancers used. Similarly alprazolam is absorbed on oral intake to the extent of 88%. Had it been absorbed to the extent of 100% the increase in effect would have been to the extent of 12%. The fact that the increase in effect is to the extent of 200% shows that a much lower dose has been instrumental in increasing the effect to much higher levels on account of adjuvant used. (Absorption data has been taken from Goodman and Gillman, The Pharmacological Basis of Therapeutics, Publisher McGraw Hill 10^{th} international edition, (2001) pages 1963 and 1926). This is a novel concept, leading to a product for use in treatment of a disease considered untreatable earlier. This apart this process eliminates 1st pass metabolism also reducing the drug dosage and hepatotoxicity of hepatotoxic drugs on long or short term use.

We describe the process by way of two examples:

1^{st} example: Increasing the transfer rate of glipizide so that the increase in activity negates the use or lowers the dose of metformin used to treat diabetes. Glipizide when taken with certain adjuvants (compounds that increase transfer into the target organ receptor sites), acts faster than oral glipizide and due to increase in receptor site concentration leads to greater receptor occupancy and increased effect.

We reproduce parts of the Glucose Tolerance Tests conducted to show the increase in efficacy of sublingual glipizide combined with adjuvants over oral glipizide. The clinical trials were conducted in two parts. The first part consisted of the following stages.

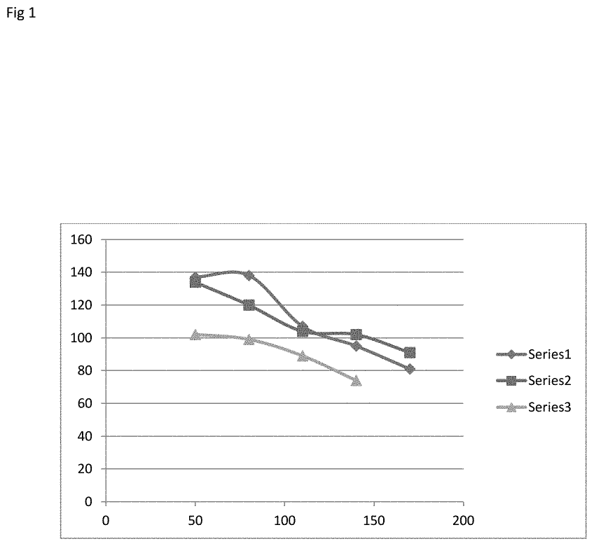

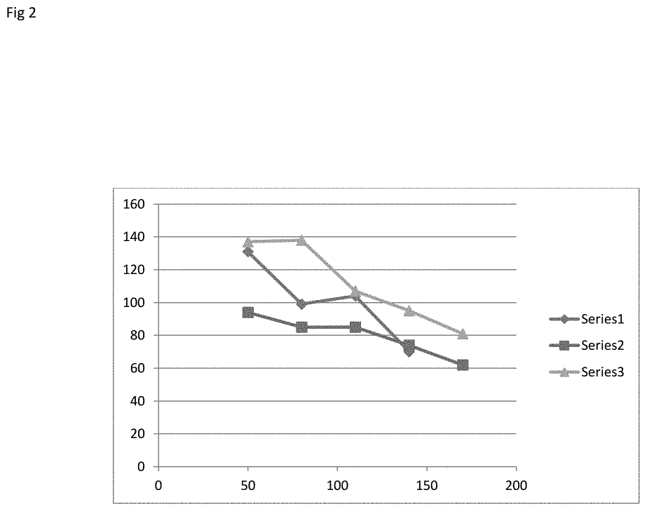

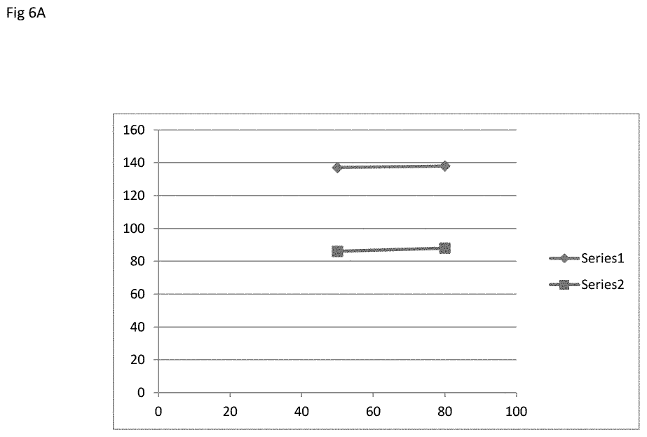

Healthy volunteers, patients with Type II D.M. and impaired glucose tolerance were chosen for the study.
1 Glucose Tolerance tests were carried out on the following lines:
a. The FBS levels of all volunteers were noted.
b. They were given placebo or oral glipizide or sub-lingual glipizide with adjuvant along with 50 to 100 gms of glucose depending on their glucose tolerance.
c. The blood sugar levels of all volunteers were monitored over different time intervals.
d. The difference in blood sugar levels between glucose and placebo and glucose and oral glipizide showed the glucose lowering effect of oral glipizide.
e. The difference in blood sugar levels of glucose and placebo and glucose and sub-lingual glipizide with adjuvant showed the effect of the latter on reduction of blood sugar levels.
f. The difference in values of (d) and (e) above showed increased efficacy of sub-lingual administration of glipizide and adjuvants over oral glipizide.
g. Two dosages of glipizide were used 2.5 mg and 5.0 mg.
h. The increase in effect was up to 32:1
i. The trials were blind and crossed over.
j. The trial results are depicted graphically in [FIGS. 1, 2 and 5.]
In the second part the effect of sublingual glipizide and adjuvant were compared to commonly marketed metformin containing anti diabetic preparations.

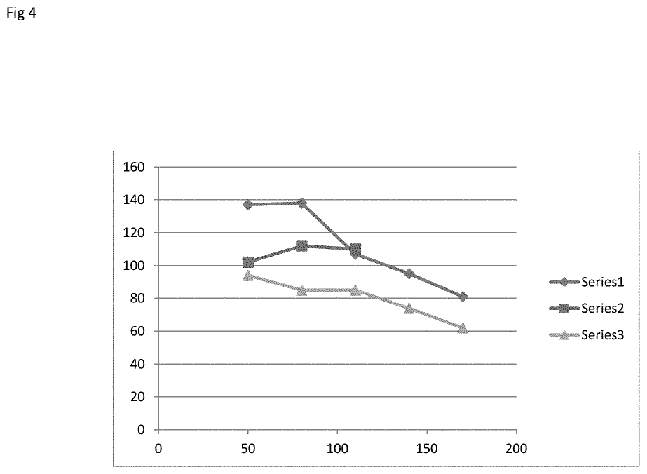

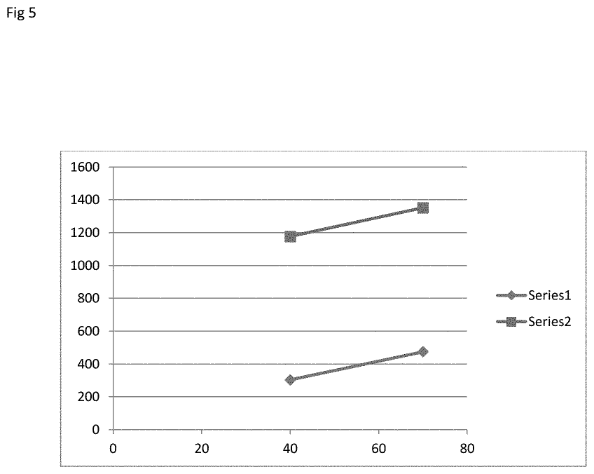

On similar lines the efficacy comparisons of sublingual glipizide with adjuvant and metformin containing combinations, glynase MF (glipizide 5 mg, metformin 500 mg), amaryl MP 2 (glimepride 2 mg metformin 500 mg and pioglitazone 15 mg) and gluconorm xl (glimepride 2 mg metformin 1000 mg) were carried out [FIGS. 3, 4 and 9]. The procedure is detailed as follows:

a. Fasting blood sugar of all the volunteers were taken and then they were given 50 to 100 gms of glucose and placebo depending on their tolerance. Their blood sugar levels were monitored till normal levels were reached. This gave the base line for neutralization of glucose by their bodies.
b. In a blind crossover study the same volunteers were administered 50 to 100 gm of glucose along with the test drugs glynase MF, amaryl MP-2 & gluconorm xl. The reduction in glucose levels due to the drugs were obtained from the difference between (a) and (b).
c. This reduction was compared to that obtained from glipizide and adjuvants. In all cases the latter proved more effective. The gluconorm-xl trial was done 11-12 hours after ingestion of the drug (details later).

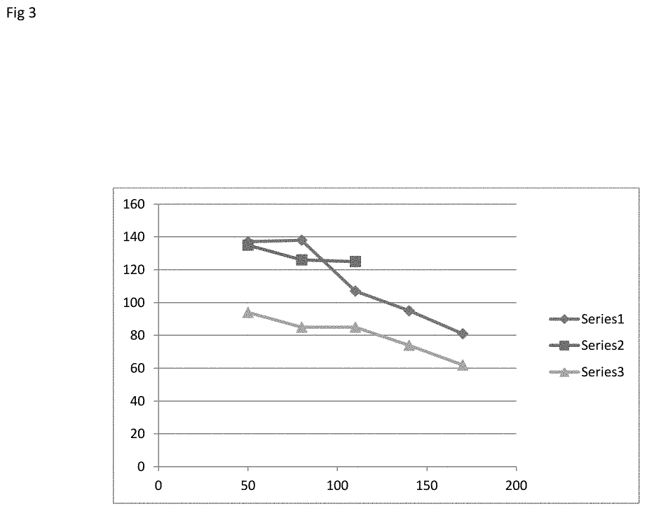

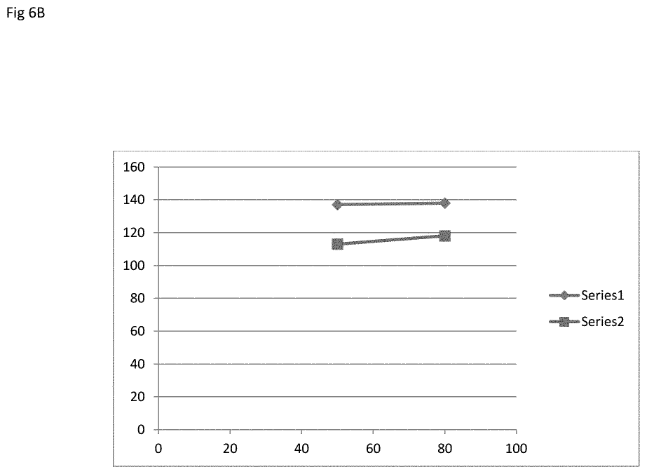

Further trials were carried out to determine the duration of action glipizide and adjuvant combination. A higher dose 7.5 mg of glipizide and permeability enhancers were used and blind crossed over glucose tolerance tests were carried out on volunteers once immediately after the ingestion of glucose and the drug [FIG. 6A] and for a 2_{nd} time 12 hours after the ingestion of the ingestion of the drug with a 2_{nd} glucose intake [FIG. 6B). The combination retained considerable activity even after 12 hrs when compared to glucose base line levels (given in FIGS. 1, 2 etc).

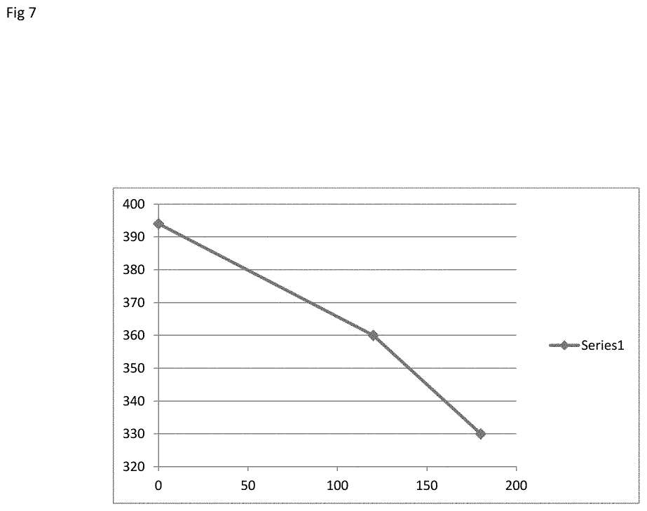

FIG. 7 shows one of the many case studies carried out. A 65 yr old female patient suffering from Type II D.M. with a FBS of 394 mg/dL (lactic acidosis and diabetic ketoacidosis levels) was treated with 7.5 mg glipizide with adjuvant. The reduction in blood sugar was 60 mg/dL in 3 hrs. This is why the product is effective in Type II D.M. patients with diabetic coma and lactic acidosis, insensitive to insulin, (high obesity levels), where a rapid reduction of blood sugar is required, risk of a stroke is imminent, and metformin is contraindicated due to its propensity to create cardiac problems. It is also useful in patients with Type I diabetes since it increases the sensitivity of cells to insulin.

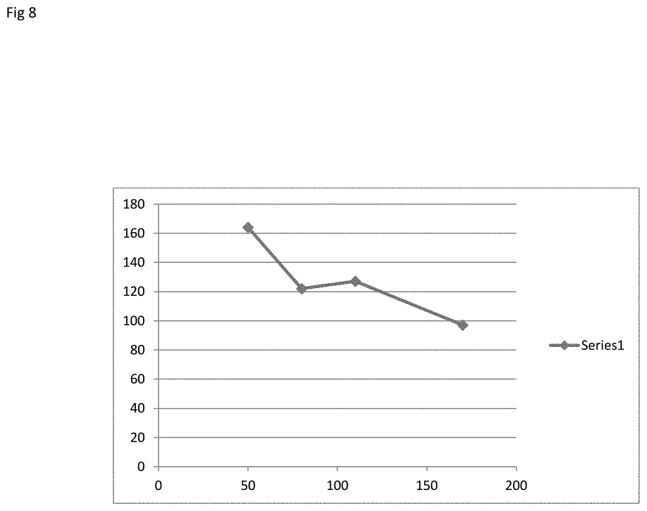

Tests were carried out using 5 mg glipizide and adjuvant with higher quantities of glucose (150 gm instead of 100 gm). It took 3 hrs to bring blood sugar back to normal. [FIG. 8]

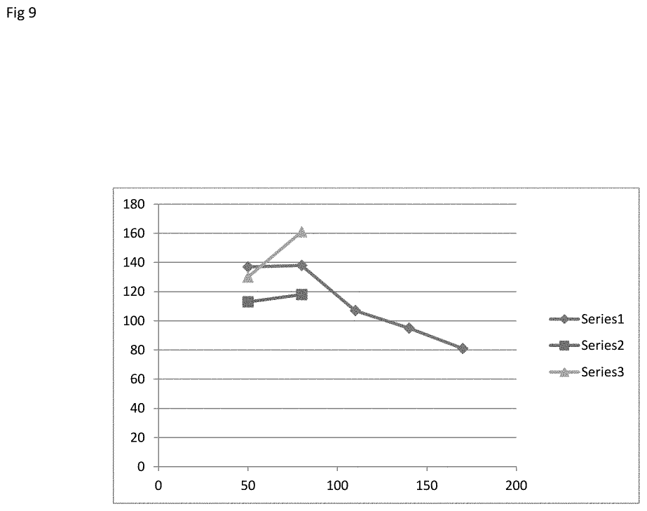

On similar lines a comparison with glimepride 2 mg and metformin 1000 mg was carried out. The drugs were administered in the morning and glucose tolerance tests carried out after 11 hr[FIG. 9]. On comparison results for glipizide and adjuvant taken after 12 hrs of drug administration proved better [FIGS. 6A&6B].

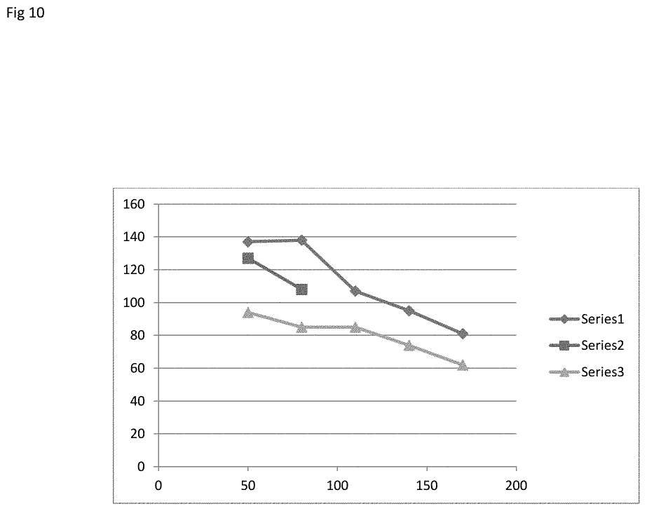

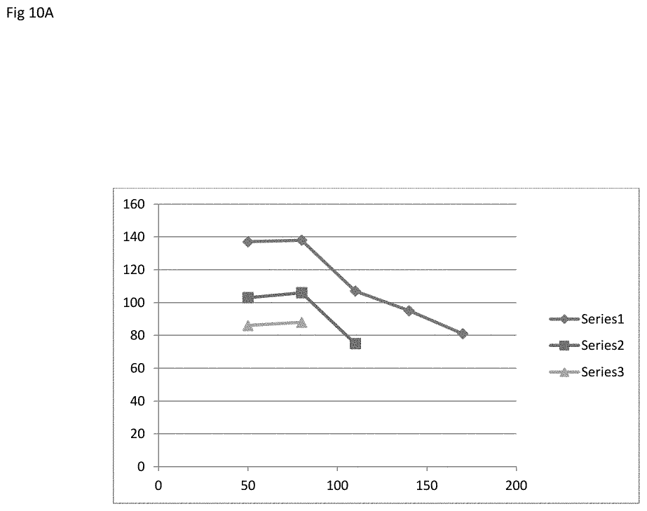

To test the efficacy of adjuvant we compared the efficacy of 5 mg of sub-lingual glipizide with adjuvant, with 5 mg of sublingual glipizide without adjuvant. The results show that 5 mg of sub-lingual glipizide with adjuvant was far more effective [FIG. 10]. Finally tests were carried out to determine whether the adjuvant used participates in receptor binding process. Keeping the amount of glipizide at 5 mg the quantity of adjuvant were doubled. In expected lines the effect was greater (FIG. 10A). This shows that the adjuvant used do not merely increase transport, had it been so the effect would not have varied with the adjuvant concentration, but actively engages in receptor binding process.

The adjuvant used in this case consisted of alcohols, oxides, peroxides, hydroxides, esters, phosphates and sulphates in the minimum ratio of 1:10 of drug to PE.

The best method for carrying out the invention: The drugs and adjuvant were punched into tablets using normal tablet making procedures. Their administration involves breaking them down in the mouth and chewing till they melt, usually 3 or 4 seconds, holding them in the mouth and swallowing them with water. Slight warmth felt after swallowing the tablets is inconsequential. Alternatively and for comatose patient a sublingual spray may be used.

Example: Glipizide—7.5 mg Adjuvant—300 mg Cyclamate Sodium—1.5 mg Methyl Cellulose 15 cps—6 mg Magnesium Stearate—3 mg Talcum—5 mg. Blend the first four ingredients add purified water to granulate. Screen, dry add talcum and magnesium stearate and punch.

2^{nd} Example: In the 2_{nd} example we show how the dose of alprazolam is almost halved by sublingual administration along with adjuvant rendering it safer and more economical.

Comparative Dosage Studies:

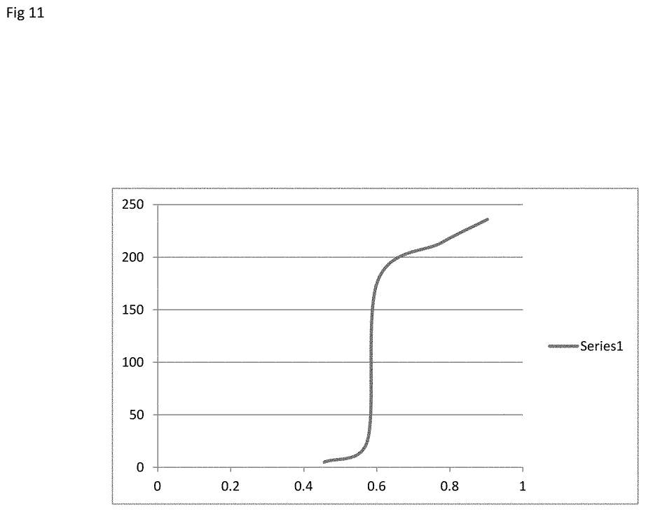

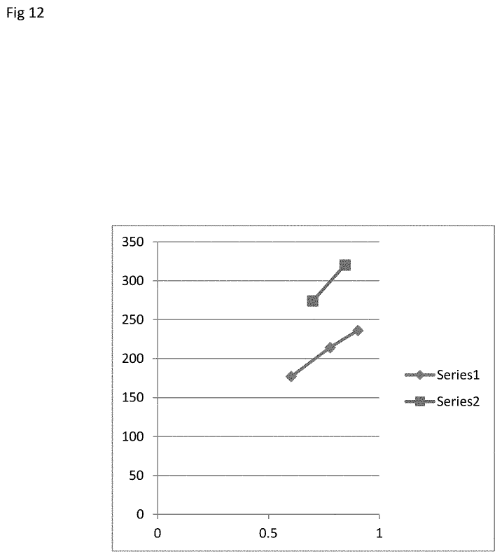

Healthy rabbits of either sex, age approximately 12 months and wt around 1.4 kg each, were selected and dosed orally with 8 mcg/kg of Alprazolam or lower. 5 doses were administered and sleep times recorded. A wash out period of 7 days were allowed between subsequent doses. A log dose response curve was plotted [FIG. 11]. The animals were then dosed sublingually with Alprazolam and adjuvant, in the same dosage range and sleep times recorded again. Comparison of effects yielded a dose equivalence ratio of (1:0.5) for oral to sublingual Alprazolam in rabbits [FIG. 12].

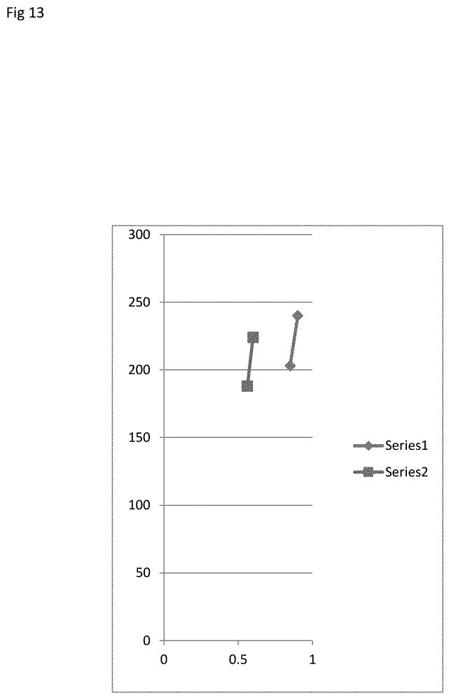

Human experiments were carried out on similar lines and a dose equivalence ratio of (1:0.55) for oral to sublingual alprazolam combined with adjuvant, was obtained [FIG. 13]

The best method for carrying out the invention: Alprazolam and adjuvant are formulated into sublingual tablets/sprays by conventional methods and administered and sleep times recorded for humans as well as animals.

Example: Alprazolam—250 mcg Adjuvant—15 mg Lactose—10 mg Talcum—1.5 mg Magnesium Stearate—500 mcg Dry Starch—1.5 mg PVP—1 mg Alcohol to granulate—q.s.
Method—Wet granulation using PVP ethanol solution as binder, talcum magnesium stearate as lubricant and dry starch as disintegrant.
The adjuvant were aldehydes ketones or sugars.

The cited examples are merely illustrative, and not exhaustive. The process may be applied to many more drugs.

Industrial applicability of this project is immense. It does away with most of the problems that plague sub-lingual administration of many drugs. I name the more important ones among them:

1 Sub-lingual administration for many drugs result in in-adequate penetration through the buccal/sublingual mucosa during normal residence time in the mouth and low organ permeability. Increasing the residence time as in buccal adhesive tablets does not help. For one, it is inconvenient to keep the preparation in the mouth for extended periods of time, and secondly the response depends not only on the amount of drug reaching the active site but also on the rate at which it is transported. Sub threshold amounts of drug being transported over extended periods of time evoke at best an inadequate response or evoke no response at all. These preparations with the help of adjuvant used ensure rapid penetration through the buccal/sub-lingual mucosa and adequate transport into the target organ/active site enabling a magnified response, so much so the dose needs to be lowered.
2 Lowered doses render the preparation less toxic compared to orally administered drugs. Since lesser amounts of drug create the same response lesser amounts need to be administered reducing the overall toxicity of the preparation.
3 Since the G I tract is bye passed and the drug is not subjected to 1st pass metabolism the product is less hepato-toxic compared to its oral counter-part.
4 These products, on a/c of reduced dosage has a lower treatment cost and are more economical compared to the oral dosage forms.

We claim:

1. A method for increasing activity of drugs on buccal/sublingual administration by increasing their receptor site concentration without increasing the dose (FIG. 10A), comprising:
An adjuvant combination capable of rapid transfer of the drug to the receptor site resulting in much higher effectiveness, rapid onset and longer duration of action, and
An active ingredient suitable for sublingual administration.

2. The method as claimed in claim 1, where the drug is glipizide, for use in the treatment of type II diabetes and diabetic ketoacidosis.

3. The method as claimed in claim 1, where the adjuvant consist of alcohols, oxides, peroxides, hydroxides, esters, phosphates and sulphates used alone or in combination used in the ratio of 1:10 minimum for glipizide to adjuvant.

4. The product obtained from the method as claimed in claim 1, consisting of glipizide and adjuvant that increase receptor site concentration of glipizide, wherein the effect of glipizide is increased 640 times compared to the maximum increase in effect conferred by membrane penetration enhancers and 3200% increase over the maximum effect of oral glipizide or sublingual glipizide without adjuvant,
To be noted here is that glipizide when orally administered is absorbed to the extent of 95% from the GI tract. Therefore had it been 100% absorbed the effect increase would have been only 5%. This 3200% increase in effect is due to increased receptor occupation of the same or similar dose of the drug due to the effect of receptor site concentration increasing adjuvant. (FIGS. 1,2,5, and 10).

5. Product obtained as claimed in claim 4, consisting of glipizide and adjuvant, which is the only small molecule treatment of diabetic ketoacidosis in Type II DM (FIG. 7) and which is more effective than metformin combinations of glipizide, glimepride, pioglitazone and such other drugs (FIGS. 3,4 and 9)

6. The method as claimed in claim 1, where the drug to be sublingually administered is alprazolam for use in treatment of anxiety and depression.

7. The method as claimed in claim 1, wherein the adjuvant consist of a group of aldehydes, ketones or sugars, selected from poly and mono saccharides, trioses, tetroses, pentoses, hexoses used alone or in combination, in the ratio of drug to adjuvant at least 1:2 or higher, for use in treatment of depression and anxiety at 50% of the dose of oral or sublingual alprazolam.

8. The product obtained from method as claimed in claim 1, consisting of alprazolam and adjuvant, that increase the receptor site concentration of alprazolam, and wherein the effect of alprazolam is increased 17 times compared to the maximum increase in effect of membrane penetration enhancers, and 200% increase over the effect of oral alprazolam or sublingual alprazolam without adjuvant.
To be noted is that alprazolam is absorbed on oral intake to the extent of 88%. Had it been absorbed to the extent of 100% the increase in effect would have been 12%. The fact that the increase in effect is 200%, shows that a much lower dose, due to the effect of the receptor site concentration increasing adjuvant, has been instrumental in raising the effect to much higher levels.
